(19) United States
(12) Patent Application Publication
(10) Pub. No.: US 2008/0241107 A1
(43) Pub. Date: Oct. 2, 2008

(54) Methods and Compositions For Preparing Pancreatic Insulin Secreting Cells

(76) Inventors: John A. Copland III, Ponte Vedrea Beach, FL (US); Randall J. Urban, Friendswood, TX (US); Yvonne H. Bodenburg, Dickinson, TX (US)

Correspondence Address: FULBRIGHT & JAWORSKI L.L.P., 600 CONGRESS AVE., SUITE 2400, AUSTIN, TX, 78701, US

(21) Appl. No.: 10/586,759

(22) PCT Filed: Jan. 24, 2005
(86) PCT No.: PCT/US05/02112

§ 371 Date: Jun. 3, 2008

Related U.S. Application Data
(60) Provisional application No. 60/538,660, filed on Jan. 23, 2004.

(51) Int. Cl. A61K 35/12; C12N 5/06; A61P 3/10; C12N 15/87

(52) U.S. Cl. 424/93.7; 435/375; 435/377; 435/455; 435/383; 435/366

(57) ABSTRACT

The present invention concerns the use of transdifferentiated cells to treat pancreatic diseases. More particularly, it provides methods for the culture and transdifferentiation of human cord blood, cells into insulin-secreting cells. It also concerns the endocrine hormones, such as insulin, produced by such cultures, and the use of the transdifferentiated cells in the treatment of diabetes.

This application claims priority to U.S. Provisional Patent Application U Ser. No. 60/538,660 filed on Jan. 23, 2004, which is hereby incorporated by reference in its entirety.

### BACKGROUND OF THE INVENTION

1. Field of the Invention

The present invention relates generally to the fields of stem cell culture and transdifferentiation. More particularly, it concerns methods and compositions for propagating cord blood stem cells. The invention also involves methods and compositions for transdifferentiation of cord blood stem cells into insulin-secreting cells, such as those in the pancreatic differentiation pathway. It also concerns the use of transdifferentiated cells to treat diabetes.

2. Description of Related Art

The β-cells of the islets of Langerhans in the pancreas secrete insulin in response to factors such as amino acids, glyceraldehyde, free fatty acids, and, most prominently, glucose. The capacity of normal islet β-cells to sense a rise in blood glucose concentration and to respond to elevated levels of glucose by secreting insulin is critical to the control of blood glucose levels. Increased insulin secretion in response to a glucose load prevents hyperglycemia in normal individuals by stimulating glucose uptake into peripheral tissues, particularly muscle and adipose tissue.

Individuals in whom islet β-cells function is impaired suffer from diabetes. Insulin-dependent diabetes mellitus, or IDDM (also known as Juvenile-onset or Type I diabetes), represents approximately 10% of all human diabetes. IDDM is distinct from non-insulin dependent diabetes (NIDDM) in that only IDDM involves specific destruction of the insulin producing β-cells of the islets of Langerhans. The destruction of β-cells in IDDM appears to be a result of specific autoimmune attack, in which the patient's own immune system recognizes and destroys the β-cells, but not the surrounding α-cells (glucagon producing) or δ-cells (somatostatin producing) that comprise the islet.

Treatment for IDDM is still centered around self-injection of insulin—clearly an inconvenient and imprecise solution—and thus the development of new therapeutic strategies is highly desirable. The possibility of islet or pancreas fragment transplantation has been investigated as a means for permanent insulin replacement (Lacy, 1995; Vajkoczy et al., 1995). Current methodologies use either cadaverous material or porcine islets as transplant substrates (Korbutt et al., 1997). However, significant problems to overcome are the low availability of donor tissue, the variability and low yield of islets obtained via dissociation, and the enzymatic and physical damage that may occur as a result of the isolation process (reviewed by Secchi et al., 1997; Sutherland et al., 1998). In addition are issues of immune rejection and current concerns with xenotransplantation using porcine islets. The recent clinical experience of islet cell transplantation is reviewed by Bretzel et al. (2001) and Oberholzer et al. (1999).

There is increasing interest in the use of stem cells for the treatment of diabetes. Peck et al. (2001) propose that pancreatic stem cells be used as building blocks for better surrogate islets for treating Type I diabetes. WO 00/47721 reports methods of inducing insulin-positive progenitor cells. WO 01/39784 reports pancreatic stem cells isolated from islet cells that are nestin-positive. WO 01/77300 reports human pancreatic epithelial progenitors that are proposed to have the capacity to differentiate into acinar, ductal, and islet cells. Deutsch et al. (2001) describe a bipotential precursor population for pancreas and liver within the embryonic endoderm. Zulewski et al. (2001) describe multipotential nestin-positive stem cells isolated from adult pancreatic islets that differentiate into endocrine, exocrine, and hepatic phenotypes. U.S. Pat. No. 6,326,201 reports pancreatic progenitor cells made by dissociating and culturing cells from pancreatic duct.

Developmental work has been done in several institutions to capitalize on the promise of pluripotent stem cells from the embryo to differentiate into other cell types. Cells bearing features of the islet cell lineage have reportedly been derived from embryonic cells of the mouse. For example, Lumelsky et al. (2001) report differentiation of mouse embryonic stem cells to insulin-secreting structures similar to pancreatic islets. Soria et al. (2000) report that insulin-secreting cells derived from mouse embryonic stem cells normalize glycemia in streptozotocin-induced diabetic mice. Regrettably, the mouse model of embryonic stem cell development does not yield strategies for differentiation that are applicable to other species. In fact, pluripotent stem cells have been reproducibly isolated from very few other mammalian species. Thomson et al. (1998) isolated embryonic stem cells from human blastocysts; and human embryonic germ (hEG) cell lines were isolated from fetal gonadal tissue (Shambloft et al. 1998). Unlike mouse embryonic stem cells, which can be kept from differentiation simply by culturing with Leukemia Inhibitory Factor (LIF), human embryonic stem cells must be maintained under very special conditions (U.S. Pat. No. 6,200,806; WO 99/20741; WO 01/51616).

It is clear that there remains a critical need to establish alternatives to the treatment of diabetes by self-injection of insulin. While stem cell research has shown promise in this regard, there is a need for improved procedures for isolating, culturing, and transdifferentiating these cells if they are to be successfully used in the treatment of diabetes.

### SUMMARY OF THE INVENTION

The present invention is based on the discovery that human cord blood stem cells can be isolated, expanded in culture, and induced to differentiate into insulin-producing cells. This discovery provides novel methods for the treatment of diabetes. The use of cord or placental blood as a source of mononuclear cells is advantageous to many other sources of stem cells known in the art because it can be obtained relatively easily and without trauma to the donor. In addition, the invention provides conditions that allow the expansion of stem cells in culture, which will further the commercial viability of the invention by providing large populations of highly pure cells for transplantation.

Thus, the present invention provides compositions and methods for expanding stem or progenitor cells in culture, particularly such cells from cord blood, and for transdifferentiating them into insulin-secreting cells. The expanded stem or progenitor cells can be used for research, diagnostic, or therapeutic applications. The transdifferentiated cells can also be used for research, diagnostic, or therapeutic purposes.

Cells that can be used according to methods and compositions of the invention include, but are not limited to, CD34+ cells (cells expressing CD34 on their surface), undifferentiated cells, stem cells, progenitor cells, cord blood cells, placental cells, neonatal or fetal cells, immature cells, pluripotent cells, and totipotent cells. The term “stem cell” is used according to its ordinary meaning, for example, as described by the National Institutes of Health (on the World Wide Web at stemcells.nih.gov). Stem cells 1) are “capable of dividing and renewing themselves for long periods”; 2) are unspecialized; and, 3) can give rise to specialized cell types.

The invention specifically contemplates the use of embryonic stem cells, adult stem cells, or neonatal and fetal stem cells. An adult stem cell typically refers to a stem cell from a particular organ or tissue that is capable of differentiating into one or more cells of that organ or tissue. Umbilical cord blood contains stem cells that are similar to embryonic stem cells in that they are believed to be capable of being differentiated into a number of different cell types, as opposed to cell types of one particular organ or tissue. Umbilical cord blood refers to blood that remains in the umbilical cord and placenta following birth and after the cord is cut. “Placental blood” is understood to be synonymous with cord blood; similarly, cord blood stem cell is considered synonymous with placental or placental blood stem cell. The use of stem cells from umbilical cord blood is specifically contemplated in certain embodiments of the invention. In some but not all cases, the use of other stem cells in specifically not considered part of the invention, particularly the use of pancreatic/endocrine progenitor or stem cells is not considered for use with some embodiments.

It will be understood that cultures or samples containing cells discussed above are also contemplated for use according to methods and compositions of the invention.

Furthermore, cells of the invention may be characterized by cell surface antigens. In some embodiments, cells used according to the invention initially express CD34+ (expression may be sustained or it may be eliminated as a cell becomes transdifferentiated). Cells may also express one or more of the following cell surface markers selected from the group consisting of: CD10, CD29, CD44, CD54, CD90, SH2, SH3, SH4, OCT-4, and ABC-p. In some embodiments, cells do not express one or more of the following cell surface markers selected from the group consisting of: CD38, CD45, SSEA3, and SSEA4.

The present invention concerns methods involving obtaining a particular cell that does not produce insulin and incubating it under certain conditions that induce the cell to produce insulin, which can be secreted. In some embodiments, there are methods for producing a cell that secretes insulin (insulin-secreting cell), as well as methods for producing a transdifferentiated cell, which refers to a cell of one type being converted into a different cell type. It will be understood that transdifferentiation of a cell includes differentiation of a cell as well. In some cases, a cell becomes transdifferentiated into a pancreatic cell. In specific embodiments, the cell becomes a pancreatic islet cell or a pancreatic beta cell.

It is contemplated that the cell produces insulin from cell's genomic insulin gene, in contrast to any cell recombinantly engineered to express insulin. Consequently, in some embodiments, the cells used in the invention have not been transformed or are not the progeny of a transformed cell (such that the progeny are also recombinantly affected). Alternatively, in some cases, cells of the invention have not been recombinantly engineered to produce insulin, but has been recombinantly engineered to prevent an immune reaction in a host after administration of the cells.

According to the invention, cells are incubated under conditions in which they are exposed to a high concentration or level of glucose. “High glucose” will refer to a glucose concentration that is higher than the concentration typically used for stem and progenitor cells, which is 5.6 mM (referred to as “low glucose”). Thus, it will be understood that high glucose includes concentrations of about or of at least about 5.7, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 mM or more, or any range therein. In some embodiments, the concentration of glucose is 25 mM or at least 25 mM. In some cases, the cells may also be incubated with one or more growth factors, in addition to exposure to high glucose. In specific embodiments, cells are also exposed to insulin. In specific embodiments, the cell is incubated in high glucose for at least 5 days or at least 10 days. In some cases, the cell is incubated in high glucose for 10 days. Cells incubated under these conditions may have been derived from cells that were CD34+ (that is, they are progeny of CD34+ cells) but have since lost CD34 expression while in culture in low glucose or upon exposure to high glucose.

It is also contemplated that cells may be incubated under these conditions, or ones described below for at least 12 hours, 24 hours, or 1, 2, 3, 4, 5, 6, 7, 8, 9, 10, 11, 12, 13, 14, 15, 16, 17, 18, 19, 20, 21 days, or 1, 2, 3, 4, 5 weeks, or 1, 2, 3, 4, 5, 6 months or more, and any range derivable therein.

Moreover, methods of the invention may include confirming that insulin is produced from the cells.

Other methods of the invention concern propagating the cells described in the previous paragraphs. The term “propagate” is used according to its ordinary meaning in the field of cell culture to mean “to multiply.” The terms “proliferate” and “expand” are used to refer to increasing the number of cells through cell division. Propagating cells may include, in certain embodiments, passaging the cells. In specific embodiments, methods are provided for propagating a CD34+ cell, a stem or progenitor cell, or a cord blood cell. It will be understood that cultures containing such cells can be used in methods and compositions of the invention.

Methods of the invention involve incubating the cell under certain conditions to sustain the cell and allow it to multiply. Such conditions include exposing the cell to insulin. In some cases, cells are exposed to lipoprotein, such as low density lipoprotein. In specific embodiments, cells are exposed to both insulin and lipoprotein. In certain embodiments, other lipoproteins such as high density lipoprotein (HDL), lipoprotein (a), and/or very low density lipoprotein (VLDL) may be used. In certain embodiments, the cell is exposed to a concentration of insulin and/or lipoprotein sufficient to promote transdifferentiation of the cell into an insulin-producing cell. Concentrations of insulin to which the cell is exposed are about or at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more μg/ml. In specific embodiments, the concentration of insulin is about 10 μg/ml. The insulin may be obtained from any source, including bovine pancreases, or it may be recombinantly produced. In certain embodiments, human insulin may be used. The concentration of lipoprotein to which the cells are exposed are about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50 or more μg/ml.

The cell may also be incubated with one or more of the following: low glucose (5.6 mM or lower), BSA, transferrin, antibiotic, or reducing agent. In specific embodiments, media is supplemented with BIT 9500 Supplement. It can be added at a ratio of about 1:5 (supplement:media). The cell is in DMEM in some embodiments of the invention, though other media can certainly be used in the context of the invention. The cell may also be exposed to one or more growth factors including, but not limited to, stem cell factor, TPO, IL-3, Flt-3, and LIF. In certain embodiments, the cell is exposed to stem cell factor, TPO, IL-3, Flt-3, and/or LIF at a concentration sufficient to promote the cell to transdifferentiate into an insulin-producing cell. In certain embodiments the cell is exposed to two or more, three or more, four or more, five or more, six or more, or all of lipoprotein, insulin, stem cell factor, TPO, IL-3, Flt-3, and LIF. In one embodiment, the cell is incubated in low glucose DMEM with BSA, insulin, transferrin, penicillin and streptomycin, low density lipoprotein, and β-mercaptoethanol, in addition to stem cell factor, TPO, IL-3, Flt-3, and LIF. Growth factors may be added to media at a concentration of about or at least about 1, 2, 3, 4, 5, 6, 7, 8, 19, 10, 11, 12, 13, 14, 15, 16, 17, 18, 19, 20, 25, 30, 35, 40, 45, 50, 55, 60, 65, 70, 75, 80, 85, 90, 95, 100 or more ng/ml. Alternatively about 0.1, 0.5, 1, 2, 3, 4, 5, 6, 7, 8, 9, 10 U may be added. The concentration of transferrin may be about or at least about 10, 20, 30, 40, 50, 60, 70, 80, 90, 100, 150, 200, 250, 300, 350, 400, 450, 500 or more μg/ml, or any range derivable therein.

Methods of the invention are effective for increasing the number of cells 2, 3, 4, 5, 6, 7, 8, 9, 10, 20, 30, 40, 50, 100, 200, 300, 400, 500, 600, 700, 800, 900, 1000, 2000, 3000, 4000, 5000, 6000, 7000, 8000, 9000, 10000 or more times (fold) as compared to the initial number of cells. Resulting cells may still express CD34, though some or all of them may have lost CD34 expression.

In further embodiments of the invention, methods also include obtaining cells to be used as part of the invention. In some cases, methods involve isolating or concentrating such cells prior to incubating them under conditions described above. A sample containing the cells may first be obtained, such as cord blood, and then subsequent steps performed on the sample. In some embodiments, methods include one or more of the following steps: concentrating leukocytes from the sample; enriching cells; and selecting cells based on expression of a cell surface marker. In specific embodiments, CD34+ cells are selected.

It will be understood that any combination of methods may be combined as part of the invention. Similarly, any steps of the inventions may be combined. Thus, in some embodiments of the invention, methods include propagating the cells and then transdifferentiating them or inducing them to secrete insulin.

It is further contemplated that methods of the invention can also include steps for recombinantly engineering the cell to reduce or prevent an immune response that might otherwise occur when it is administered to a patient. In some cases, the cell is recombinantly engineered to reduce or prevent the presence or expression of one or more cell surface proteins on the cell. Cell surface proteins that may be targeted are human leukocyte antigen (HLA) proteins, such as HLA-A, HLA-B and HLA-DR proteins. The intention is to reduce the risk of rejection in a patient who receives cells from another person.

It is contemplated that the invention further concerns compositions comprising such cells under the conditions described above. Compositions include any of the cells described above under the conditions described above. Specific embodiments include stem or progenitor cells, CD34+ cells, undifferentiated cells, and cord blood cells in media containing insulin and/or lipoprotein. Other compositions specifically include compositions comprising an insulin-secreting cell in a high glucose media containing insulin and/or lipoprotein. It is contemplated that the insulin-secreting cell can be produced according to methods of the invention.

Methods and compositions of the invention for propagating cells and inducing insulin secretion or production can be used for methods of treating diabetes in a patient. It is understood that treatment may be applied to humans. In some embodiments, the patient is administered an effective amount of transdifferentiated cells producing insulin. In some cases, cells are autologous, while in others, the cells are heterologous with respect to the recipient. Thus, it is contemplated that the patient may also be administered one or more immunosuppressing agents. Furthermore, cells may have been engineered to reduce or prevent expression of one or more cell surface proteins.

The diabetes that is treated can be any form, though treatment of Type I and Type II are specifically contemplated. The number of cells that can be administered to the patient can be about or at least about 1, 2, 3, 4, 5, 6, 7, 8, 9×10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more, or any range derivable therein.

Methods of the invention also include methods of screening using compositions described above. Cells may be exposed to a candidate substance and the ability of the candidate substance to alter the phenotype, such as insulin production, may be tested.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B. Growth curves of two stem cell populations (Isolate #1 and Isolate #2 in FIG. 1A and FIG. 1B, respectively) isolated from human cord blood based on expression of CD34 using immunomagnetic beads show that the cells expand rapidly in low glucose DMEM containing: BIT 9500 supplement (1:5 media ratio), Pen/Strep (1:100), Low Density Lipoprotein (20 ug/ml), β-Mercaptoethanol (0.1 mM), Stem Cell Factor (50 ng/ml), TPO (10 U/ml), IL 3 (10 ng/ml), Flt-3 (10 ng/ml), and LIF (10 ng/ml). As shown in FIG. 1A, Isolate #1 doubled every 1.7 days between days 0-5, and doubled every 1.5 days between days 5-7. Isolate #2 doubled every 1.5 days between days 0-5, and doubled every 22 hours between days 5-7 (FIG. 1B).

FIG. 2A and FIG. 2B. Immunohistochemical analysis of cord blood stem cells exposed to high glucose produced insulin protein (FIG. 2A), whereas cells that have been grown in normal culture media did not produce insulin protein (FIG. 2B).

FIG. 3. RT-PCR analysis demonstrates that insulin mRNA is present in human cord blood stem cells exposed to high glucose for 10 days in cell culture. 20 μl of PCR product was run on a 1.75% agarose TBE gel and stained with ethidium bromide. Lane 1, ladder; Lane 2, empty; Lane 3, positive control; Lane 4, human cord blood stem cells/10 days high glucose; Lane 5, human cord blood stem cells/10 days without glucose; Lane 6, human cord blood stem cells/7 days in low glucose. The expected size of the PCR product is 331 base pairs in length.

FIG. 4A-E. Human cord blood stem cells differentiate into insulin secreting cells. A. SC-DIF#3 with DAPI. B. SC-DIF#3 with Insulin-FITC. C. Rin cells with DAPI. D. Rin cells with Insulin-FITC. E. Light microscopy.

### DESCRIPTION OF ILLUSTRATIVE EMBODIMENTS

A stem cell is a cell that has the capacity to both self-renew and to generate differentiated progeny. Two stem cells that are already in clinical use are hematopoietic stem cells (HSCs) and mesenchymal stem cells (MSCs). Both HSCs and MSCs have been suggested to share common bone marrow precursors that express CD34 antigen. The mammalian hematopoietic system produces at least eight distinct lineages of mature blood cells in a continuous manner throughout adult life. These lineages include red blood cells, monocytic, granulocytic, basophilic, myeloid cells, the T and B cells and platelets. Complex quantitative analyses of HSCs, in some cases, demonstrated that a single transplantable stem cell is both necessary and sufficient to transfer an intact, normal hematopoietic system to a recipient host Jordan et al. (1990); Smith et al. (1991).

The proliferation and development of HSCs in vivo is promoted by contact with bone marrow stromal cells and the surrounding extracellular matrix. While there is some ability of soluble cytokines or growth factors to promote survival and proliferation of stem cells and their progeny in the absence of stromal cell matrix, the primitive HSCs can only be maintained, in the long term, when co-cultured with the appropriate stromal cell environment (Dexter et al., 1990). The characterization of CD34 antigen on HSCs, expressed only by 0.5-5% of human bone marrow cells, has enabled the purification of HSCs in commercial quantities. CD34 is not expressed on more mature counterparts (Civin et al., 1990). Using the long term bone marrow culture system, it has been established that CD34+ HSCs can survive in vitro and differentiate when allowed to grow in contact with bone marrow derived stromal cells, which produce a plethora of factors including M-CSF, GM-CSF, G-CSF, IL-1, IL-6, IL-7, TGF-beta, LIF, SCF (Heyworth et al., 1997). It has been shown that bone-marrow derived HSCs and MSCs can be directed to enter into the pancreatic differentiation pathway, as determined by the expression of the genes Isl-1, Pdx-1, Pax-4, Pax-6, Glut-2, and insulin, which are relevant to pancreas organogenesis (U.S. 2002/0182728).

The term “umbilical cord blood” or “cord blood” refers to blood obtained from a neonate or fetus, most preferably a neonate and preferably refers to blood which is obtained from the umbilical cord or the placenta of newborns. The use of cord or placental blood as a source of mononuclear cells is advantageous because it can be obtained relatively easily and without trauma to the donor. Cord blood cells can be used for autologous transplantation or allogenic transplantation, when and if needed. Cord blood is preferably obtained by direct drainage from the cord and/or by needle aspiration from the delivered placenta at the root and at distended veins.

Human cord and placental blood provides a rich source of hematopoietic stem cells. Umbilical cord blood stem cells have been used to reconstitute hematopoiesis in children with malignant and nonmalignant diseases after treatment with myeloablative doses of chemoradiotherapy (Sirchia and Rebulla, 1999). Early results show that a single cord blood sample provides enough hematopoietic stem cells to provide short- and long-term engraftment, and that the incidence and severity of graft-versus-host disease has been low even in HLA-mismatched transplants. In addition, it has been reported that cord blood can be the source of cells that can differentiate into neuronal and glial cells (U.S. Patent Application 20020028510).

The present invention demonstrates that human cord blood stem cells can be isolated, expanded in culture, and induced to differentiate into insulin-producing cells. The invention solves the problem of producing large populations of insulin-producing cells for transplantation by providing methods for expanding and transdifferentiating cord blood stem cells. For example, the cells can be expanded by culture in low glucose DMEM containing: BIT 9500 supplement at a 1:5 media ratio (final concentrations:1% BSA, 10 ug/ml Bovine Pancreatic Insulin, 200 ug/ml Human Transferrin), Pen/Strep (1:100), Low Density Lipoprotein (20 ug/ml), β-Mercaptoethanol (0.1 mM), Stem Cell Factor (50 ng/ml), TPO (10 U/ml), IL 3 (10 ng/ml), Flt-3 (10 ng/ml), and LIF (10 ng/ml). The cells can be directed to produce insulin by raising the glucose concentration of the culture media. In a specific example, the glucose concentration of the media is raised from 5.6 mM to 25 mM. The invention's culture method for transdifferentiation of cord blood stem cells to insulin-producing cells preferably utilizes the culture conditions specified herein. However, it will be obvious to those skilled in the art that various changes and modifications may be made within the spirit and scope of the invention.

The ability of a cell to produce insulin can be assayed by a variety of methods known to those of skill in the art. For example, insulin mRNA can be detected by RT-PCR or insulin may be detected by antibodies raised against insulin. In addition, other indicators of pancreatic differentiation include the expression of the genes Isl-1, Pdx-1, Pax-4, Pax-6, and Glut-2. Other phenotypic markers for the identification of islet cells are disclosed in U.S. 2003/0138948, incorporated herein in its entirety.

A stem cell, progenitor cell, or differentiated cell is “transplanted” or “introduced” into a mammal when it is transferred from a culture vessel into a patient. Transplantation, can include the steps of isolating a stem cell according to the invention and transferring the stem cell into a patient. Transplantation can involve transferring a stem cell into a patient by injection of a cell suspension into the patient, surgical implantation of a cell mass into a tissue or organ of the patient, or perfusion of a tissue or organ with a cell suspension. The route of transferring the stem cell for transplantation will be determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ. For the treatment of diabetes, preferred sites of implantation include the pancreas, the liver, under the kidney capsule, or in a subcutaneous pocket.

Transplantation, can include the steps of isolating a stem cell according to the invention, and culturing and transferring the stem cell into a patient. Transplantation, can include the steps of isolating a stem cell according to the invention, differentiating the stem cell, and transferring the stem cell into a patient. Transplantation, can include the steps of isolating a stem cell according to the invention, differentiating and expanding the stem cell and transferring the stem cell into a patient.

The treatment methods of the invention include the implantation of transdifferentiated cells that produce insulin into individuals in need thereof. The invention provides a method of controlling or eliminating a diabetic (IDDM) patient's need for insulin therapy because the transdifferentiated cells can produce insulin in vivo. Thus, the method can be used to treat or reverse IDDM. Sites of implantation include in the liver, pancreas, under the kidney capsule or in a subcutaneous pocket. Alternatively, the endocrine hormones (especially insulin) may be harvested from the cultured transdifferentiated cells, using methods known in the art, and administered to the patient.

The appropriate cell implantation dosage in humans can be determined from existing information relating to ex vivo islet transplantation in humans, further in vitro and animal experiments, and from human clinical trials. From data relating to transplantation of ex vivo islets in humans, the number of transdifferentiated cells per patient kg can be calculated; according to the hormone production of the cells. Assuming long-term survival of the implants following transplantation, less than the number of β-cells used in ex vivo islet transplantation may be necessary. From in vitro culture and in vivo animal experiments, the amount of hormones produced can be quantitated, and this information is also useful in calculating an appropriate dosage of implanted material. Additionally, the patient can be monitored to determine adherence to normal glucose levels. If such testing indicates an insufficient response or hyperinsulinemia, additional implantations can be made.

Preferably, the transdifferentiated cells would be derived from the patient that is being treated or from a donor related to the patient so as to avoid immune rejection. Thus, no immune suppressing therapy would be required for transplantation of cells into the diabetic patient. Alternatively, where autologous cells are not available, allogenic cells could be modified to evade or suppress immune responses to ameliorate donor rejection of transplanted cells. For example, it can be useful to encapsulate the transdifferentiated cells in a capsule that is permeable to the endocrine hormones, including insulin, glucagon, somatostatin and other pancreas produced factors, yet impermeable to immune humoral factors and cells. Preferably the encapsulant is hypoallergenic, is easily and stably situated in a target tissue, and provides added protection to the implanted structure.

Protection from immune rejection can also be provided by genetic modification of the transdifferentiated cells, according to any method known in the art. For example, transdifferentiated cells could be genetically modified to eliminate the HLA markers from the cell surface or to express genes that suppress immune responses. Autoantibody and CTL resistant cells can be produced using methods such as those disclosed in U.S. Pat. No. 5,286,632; U.S. Pat. No. 5,320,962; U.S. Pat. No. 5,342,761; and in WO 90/11354; WO 92/03917; WO 93/04169; and WO 95/17911. Selection of resistant transdifferentiated cells may be accomplished by culturing these cells in the presence of autoantibody or IDDM associated CTLs or CTLs activated with IDDM specific autoantigens. As a result of these techniques, cells having increased resistance to destruction by antibody or T-lymphocyte dependent mechanisms may be generated. Likewise, the human leukocyte antigen (HLA) profile of the transdifferentiated cell can be modified, optionally by an iterative process, in which the transdifferentiated cell is exposed to normal, allogenic lymphocytes, and surviving cells selected.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Isolation and Culture of Stem Cells From Human Cord Blood

Isolation of stem cells from human cord blood. Human cord blood was obtained from the Ob/Gyn department at the University of Texas Medical Branch at Galveston. The cord blood was collected in either a sterile heparinized bag or in a tube containing ACD- at the AABB recommend a ratio of 1:7 (1 part ACD-A solution to 7 parts whole blood). The cord blood was used within 4-6 hours of collection.

Enrichment of cord blood progenitor cells. To concentrate leukocytes, 2 ml of HetaSep was added per 10 ml blood at room temperature in a 50 ml centrifuge tube, mixed well, and centrifuged for five minutes at 50×g. The supernatant plus approximately the top 10% of pellet volume was removed and retained. The remainder of the pellet was discarded. The 50 ml centrifuge tube was then filled with wash medium, PBS+0.5% BSA (without Ca²⁺ and Mg²⁺), the pellet resuspended, and then centrifuged at 300×g for 10 minutes. The supernatant was removed and discarded. The pellet was resuspended in wash medium by adding 0.5 ml of wash medium per 10 ml of the original cord blood volume.

The progenitor cells were further enriched using RosetteSep™. Briefly, 75 μl of RosetteSep™ cocktail was added per 10 ml of original cord blood volume, mixed well, and incubated at room temperature for 10 minutes. Next, the sample was diluted with approximately 2× volume of wash medium and mixed gently. The diluted sample was then layered on top of Ficoll-Paque at a 1:2 dilution, and then centrifuged for 20 minutes at 1200×g at room temperature. Enriched cells were then removed from the Ficoll-Paque plasma interface. The enriched cells were then washed with wash medium and then centrifuged for 10 minutes at 1200 RPMs. The supernatant was removed prior to ammonium chloride (NH₄Cl) lysis.

Residual red blood cells were removed by lysis with NH₄Cl. Briefly, cells were transferred to a 15 ml tube and resuspended in a 4:1 volume of NH₄Cl solution (4 ml NH₄Cl to 1 ml sample). The cell suspension was vortexed and then left at room temperature for 5 minutes or placed on ice for 10 minutes. The cells were then twice washed in wash medium and centrifuged at 300×g for 8-10 minutes.

Next, CD34⁺ cells were enriched using EasySep™. The sample was transferred to a microcentrifuge tube and centrifuged at 6000 RPMs for 1 minute. The cells were resuspended at a concentration of 2×10⁸ cells/ml in PBS+0.5% BSA+1 mM EDTA (without Ca²⁺ and Mg²⁺). EasySep™ Positive Selection Cocktail was added at 200 uL/ml cells, mixed well, and incubated at room temperature for 15 minutes. EasySep™ Magnetic Nanoparticles were then added to the cell suspension and incubated at room temperature for 10 minutes. The total volume of the cell suspension was then brought to 2.5 mL by adding PBS+0.5% BSA+1 mM EDTA (without Ca²⁺ and Mg²⁺). The cell suspension was mixed in the tube by gently pipetting up and down 3-4 times. The cap was removed from the tube and the tube was placed into the magnet. After 10 minutes, the magnet and the tube were inverted and the supernatant fraction allowed to pour off. The tube was removed from the magnet and 2.5 mL of PBS+0.5% BSA+1 mM EDTA (without Ca²⁺ and Mg²⁺) was added. The cell suspension was mixed by gently pipetting up and down 3-4 times. The tube was placed back in the magnet for ten minutes. This process was repeated for a total of 2-3 10 minute separations in the magnet. After the final separation in the magnet, the positively selected cells were resuspended in 1 ml of PBS+0.5% BSA+1 mM EDTA (without Ca²⁺ and Mg²⁺) and the cells were counted using a hemocytometer.

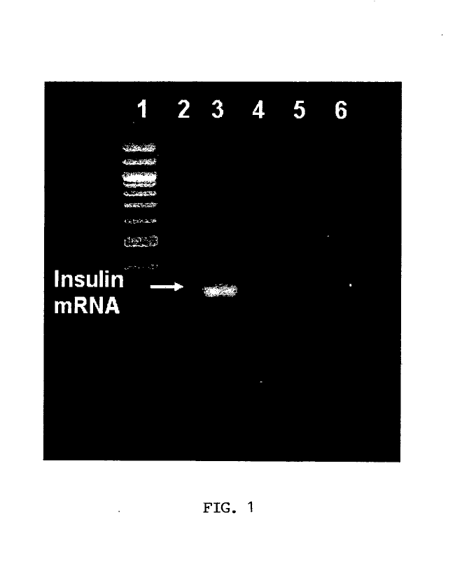

Stem cell culture. Cells were plated in sterile 6-well culture dishes at 10-7.5×10⁴ cells per 2 ml media in low glucose DMEM containing: BIT 9500 supplement at a 1:5 media ratio (final concentrations:1% BSA, 10 ug/ml Bovine Pancreatic Insulin, 200 ug/ml Human Transferrin), Pen/Strep (1:100), Low Density Lipoprotein (20 ug/ml), β-Mercaptoethanol (0.1 mM). In addition, the following cytokines were added to the media: Stem Cell Factor (50 ng/ml), TPO (10 U/ml), IL 3 (10 ng/ml), Flt-3 (10 ng/ml), LIF (10 ng/ml). These culture media conditions enabled the rapid expansion of the stem cells (FIG. 1A and FIG. 1B).

Glucose induction of stem cells. The glucose concentration of the media was raised from 5.6 mM to 25 mM by adding 70 μl of a 10% glucose solution (554 mM) to 2 ml of media.

Freezing cells. The stem cells can be frozen in liquid nitrogen at 1×10⁶ cells per ml of media with 10% DMSO.

For RNA and protein analysis, cell pellets can be frozen at −70° C. Briefly, cells are pelleted in microcentrifuge tubes and washed 2 times in PBS and then immediately frozen at −70° C.

### EXAMPLE 2

### Insulin Synthesis in Glucose-Induced Stem Cells

Human cord blood stem cells isolated from fresh cord blood based on expression of CD34 using immunomagnetic beads were induced to express insulin by exposure to high glucose concentrations. The cells were expanded in low glucose media and then put into media containing high glucose as described in Example 1. After 10 days in the high glucose media, insulin synthesis was verified by immunohistochemical and RT-PCR analysis. The immunohistochemical and RT-PCR analyses verified insulin synthesis in cells exposed to high glucose, whereas control stem cells did not express insulin.

Immunohistochemistry. Cells were washed twice with PBS before being resuspended at 200,000 cells/100 μl PBS. Cells were cytospun for 5 minutes at 500 RPMs. Slides were stored at −20° C. until ready to fix and stain.

Slides were fixed with 4% paraformaldehyde (500 μl) at room temperature for 20 minutes, and then washed twice with PBS. Slides were then fixed with 95% ethanol for 5 minutes and washed three times with PBS. Next, the slides were blocked for 30 minutes in 2% Milk/0.1% Triton/PBS at room temperature. The slides were then washed with 1% BSA/PBS.

The slides were incubated in Primary antibody (Anti-Insulin Monoclonal, Sigma #1-2018, Dilution 1:400) overnight at 4° C. (in dark) with gentle rocking. The slide was then washed 2 times with 1% BSA/PBS for 15 minutes. The FITC-labeled secondary antibody (Anti-Mouse IgG FITC, Vector Laboratories #FI2000, Dilution 1:500) was then added and incubated for 1 hour at 4° C. in dark.

Following incubation with the secondary antibody, the slides were washed (3×) with PBS for 5 minutes. Excess fluid was removed and 1 drop of Vectashield® mounting media with DAPI was added and the coverslip was carefully placed on top.

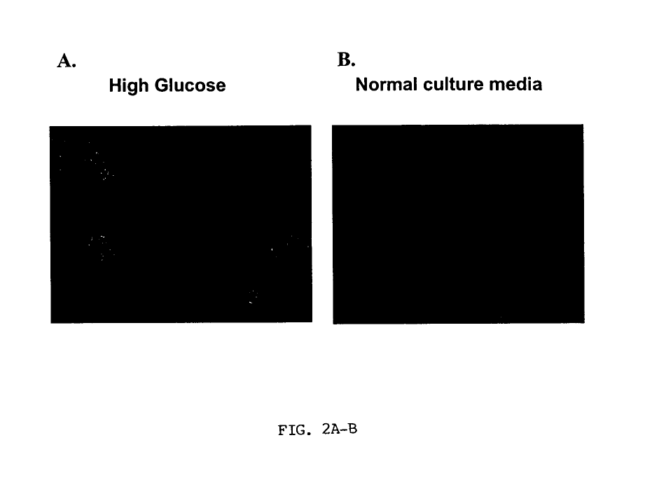

As shown in FIG. 2A, immunohistochemical analysis of cord blood stem cells exposed to high glucose produced insulin protein, whereas cells that were grown in normal culture media did not produce insulin protein (FIG. 2B).

RT-PCR. RNA was isolated using Tri Reagent (Sigma). cDNA was made using 1 μg of total RNA with SuperScript™ First-Strand Synthesis System for RT-PCR using Oligo(dT) (Invitrogen). Next, target cDNA was amplified using Platinum™ PCR Supermix (Invitrogen) with specific human insulin primers (Forward 5′-ATGGCCCTGTGGATGCGCCT-3′ (SEQ ID NO:1) and Reverse 5′-TAGTTGCAGTAGTTCTCCAGC-3′ (SEQ ID NO:2)) and 2 μl of cDNA reaction using the following thermal cycles: 1 cycle at 94° for 2 min; 35 cycles of 94° for 15 sec, 55° for 30 sec, 72° for 1 min; and 1 cycle at 72° for 7 min. 20 ul of PCR product was run on a 1.75% agarose TBE gel. The expected PCR product is 331 base pairs in length.

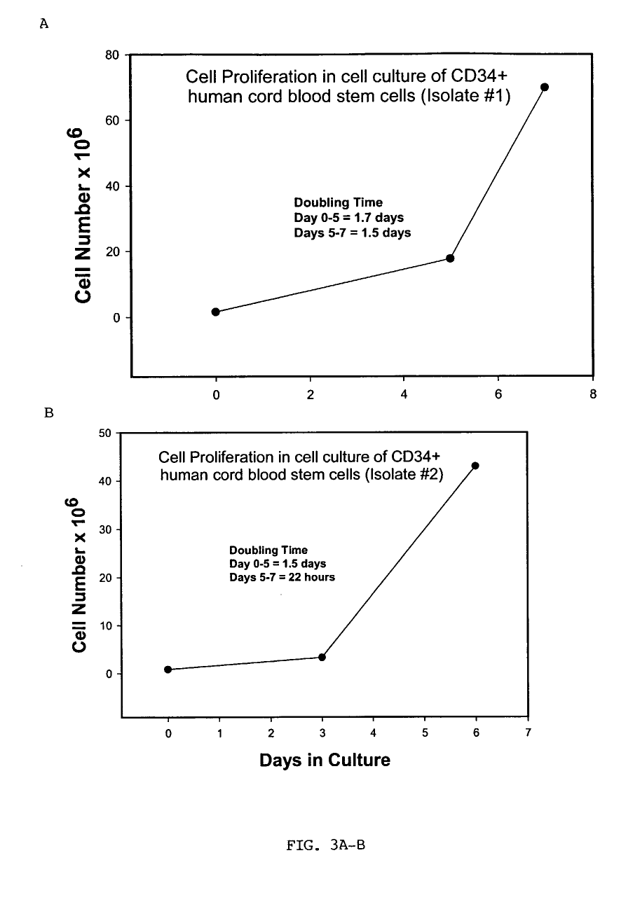

As shown in FIG. 3, purified human cord blood stem cells exposed to high glucose levels for 10 days in culture synthesize insulin mRNA (lane 4). Cells not exposed to high glucose do not synthesize insulin mRNA (FIG. 3, lanes 5 and 6). Rin cells, which are known to synthesize insulin, were used as a positive control (FIG. 3, lane 3).

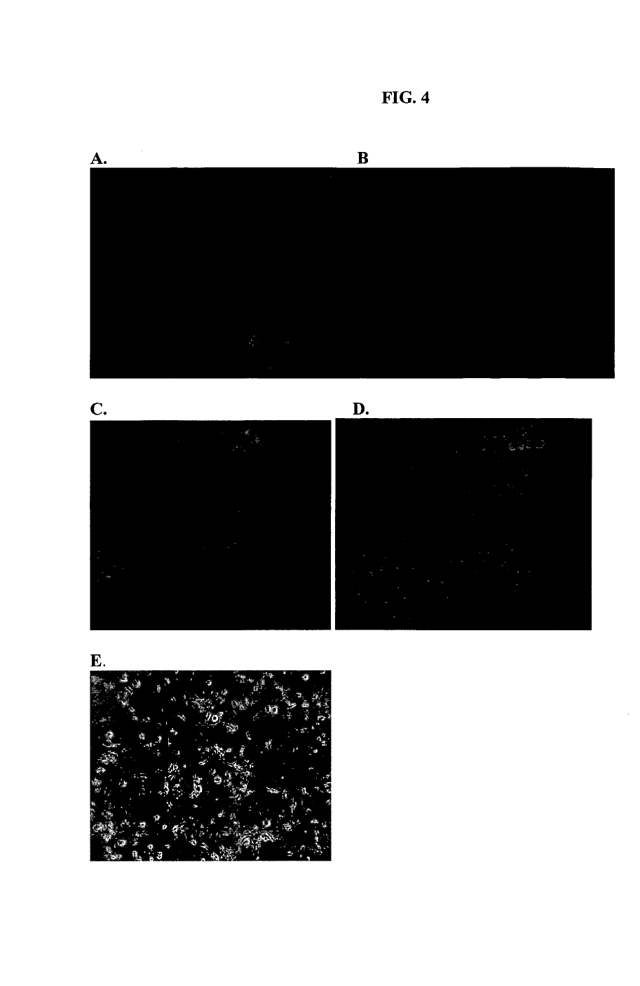

Further experiments using Immunocytochemical (ICC) analysis demonstrated insulin protein expression in differentiated human cord blood cells as described above. B. Cells from FIG. 4A are stained with DAPI to demonstrate that these are viable cells. Each blue spot represents the nucleus of a cell. Comparing FIG. 4A to FIG. 4B, it will be noted that each blue spot (cell nucleus) corresponds with a green spot (insulin) demonstrating that all cells are producing insulin. Rin cells are used as a positive control for insulin expression. These cells stain brightly green demonstrating that cells are producing insulin (FIG. 4C). DAPI staining of cells in FIG. 4C demonstrates that an intact nucleus is present and that all cells are viable and producing insulin (FIG. 4D). Light microscopy shows the cell morphology of differentiated insulin producing human cord blood cells after 25 days in culture (FIG. 4E).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Pat. No. 5,286,632
U.S. Pat. No. 5,320,962
U.S. Pat. No. 5,342,761
U.S. Pat. No. 6,200,806
U.S. Pat. No. 6,326,201
U.S. Appln. 2002/0182728
U.S. Appln. 20020028510
Bretzel et al. Exp. Clin. Endocrinol. Diabetes, 190(Suppl. 2):S384, 2001.
Civin et al., Prog. Clin. Biol. Res., 333:387-401, 1990.
Deutsch et al., Development, 128:871, 2001.
Dexter et al. Ciba Found. Symp., 148:76-86, 1990.
Heyworth et al., In: Stem Cells, Academic Press Ltd., 243-441, 1997.
Jordan et al., Genes Dev., 4:220-232, 1990.
Korbutt et al., Ann NY Acad Sci., 831: 294-303, 1997.
Lacy, Hosp. Pract., 30(6): 41-45, 1995.
Lumelsky et al., Science, 292:1389, 2001.
Oberholzer et al., Ann. N.Y. Acad. Sci., 875:189, 1999.
PCT Appln. WO 00/47721
PCT Appln. WO 01/39784
PCT Appln. WO 01/51616
PCT Appln. WO 01/77300
PCT Appln. WO 90/11354
PCT Appln. WO 92/03917
PCT Appln. WO 93/04169
PCT Appln. WO 95/17911
PCT Appln. WO 99/20741
Peck et al., Ann. Med., 33:186, 2001.
Secchi et al., Diabetologia., 40(2): 225-231, 1997.
Shambloft et al., Proc. Natl. Acad. Sci. USA, 95:13726, 1998.
Sirchia and Rebulla, Haematologica, 84:738-747, 1999.
Smith et al., Proc. Natl. Acad. Sci. USA, 88:2788-2792, 1991.
Soria et al., Diabetes, 49:157, 2000.
Sutherland et al., Transplant Proc., 30(5): 2264-2266, 1998.
Thomson et al., Science, 282:114, 1998.
Vajkoczy et al., Transplantation, 60(2): 123-127, 1995.
Zulewski et al., Diabetes, 50:521, 2001.

1. A method for producing a cell that secretes insulin comprising:
a) obtaining a cell that does not produce insulin; and,
b) incubating the cell with media containing high glucose, wherein the cell secretes insulin.

2. The method of claim 1, wherein the cell is transdifferentiated into an insulin-producing cell.

3. The method of claim 2, wherein the cell is transdifferentiated into a pancreatic cell.

4. The method of claim 3, wherein the cell is transdifferentiated into a pancreatic beta cell.

5. The method of claim 1, wherein the cell that does not produce insulin is an undifferentiated cell.

6. The method of claim 5, wherein the undifferentiated cell is a human cell.

7. The method of claim 6, wherein the cell was obtained from cord blood or tissue.

8. The method of claim 6, wherein the cell was obtained from placental blood or tissue.

9. The method of claim 6, wherein the human cell is an autologous cell.

10. The method of claim 5, wherein the undifferentiated cell is a stem or progenitor cell.

11. The method of claim 6, wherein the undifferentiated cell is a multipotent cell, a totipotent cell, or a hematopoietic cell.

12. The method of claim 11, wherein the undifferentiated cell is a hematopoietic cell.

13. The method of claim 12, wherein the hematopoietic cell is capable of differentiation into a dendritic cell, a granulocyte, an erythroid cell, a monocyte, a B cell, or a T lymphocyte.

14. The method of claim 5, wherein the undifferentiated cell expresses CD34.

15. The method of claim 5, wherein the undifferentiated cell does not express CD38.

16. The method of claim 14, wherein the undifferentiated cell also expresses one or more of the following cell surface markers selected from the group consisting of: CD10, CD29, CD44, CD54, CD90, SH2, SH3, SH4, OCT-4, and ABC-p.

17. The method of claim 14, wherein the cell does not express one or more of the following cell surface markers selected from the group consisting of: CD38, CD45, SSEA3, and SSEA4.

18. The method of claim 1, wherein the media contains at least 10 mM glucose.

19. The method of claim 18, wherein the media contains at least 25 mM glucose.

20. The method of claim 1, wherein the media further comprises a lipoprotein.

21. The method of claim 20, wherein the lipoprotein is high density lipoprotein (HDL), low density lipoprotein (LDL), lipoprotein (a), or very low density lipoprotein (VLDL).

22. The method of claim 1, wherein the media further comprises at least one of β-mercaptoethanol, stem cell factor, TPO, IL-3, Flt-3, or LIF.

23. The method of claim 20, wherein the media further comprises at least one of β-mercaptoethanol, stem cell factor, TPO, IL-3, Flt-3, or LIF.

24. The method of claim 6, wherein the media further comprises at least one additional compound in an amount sufficient to promote transdifferentiation of the undifferentiated cell into the cell that secretes insulin.

25. The method of claim 24, wherein the additional compound is a lipoprotein.

26. The method of claim 25, wherein the lipoprotein is low density lipoprotein.

27. The method of claim 6, wherein the additional compound is low density lipoprotein, β-mercaptoethanol, stem cell factor, TPO, IL-3, Flt-3, or LIF.

28. The method of claim 6, wherein the media further comprises low density lipoprotein, β-mercaptoethanol, stem cell factor, TPO, IL-3, Flt-3, and LIF in amounts sufficient to promote transdifferentiation of the undifferentiated cell into the cell that secretes insulin.

29. The method of claim 1, wherein the cell is incubated in media containing glucose for at least 5 days.

30. The method of claim 1, wherein the media further comprises insulin.

31. The method of claim 30, wherein the insulin is present in an amount sufficient to promote transdifferentiation of an undifferentiated cell into the cell that secretes insulin.

32. The method of claim 31, wherein the insulin is human insulin.

33. The method of claim 1, further comprising propagating the cell prior to incubation in high glucose.

34. The method of claim 33, wherein the propagation comprises passaging the cell at least once.

35. The method of claim 1, further comprising recombinantly engineering the cell to reduce or prevent an immune response to the cell after the cell is administered to the patient.

36. The method of claim 35, wherein the cell is recombinantly engineered to reduce or prevent the presence of one or more cell surface protein on the cell.

37. The method of claim 36, wherein the cell surface protein is a human leukocyte antigen (HLA) protein.

38. A method for propagating a CD34+ cell or a stem or progenitor cell in culture comprising:
a) obtaining a CD34+ cell; and,
b) incubating the cell in a media comprising insulin and/or lipoprotein.

39. The method of claim 38, wherein the cell is a cord blood or placental cell.

40. The method of claim 38, wherein the number of total cells in the culture increases more than two-fold.

41. The method of claim 38, further comprising:
c) concentrating leukocytes from a cord blood sample;
d) selecting stem cells using a CD34 protein marker prior to incubating stem cells with a media comprising insulin and/or lipoprotein, wherein the number of stem cells increases.

42. A composition comprising an insulin-producing cell in a high glucose media containing insulin and/or lipoprotein.

43. The composition of claim 42, wherein the high glucose media comprises at least 25 mM glucose.

44. A method for treating diabetes in a patient comprising:
a) administering to the patient an effective amount of transdifferentiated cells producing insulin.

45. The method of claim 44, wherein the cells are derived from the patient.

46. The method of claim 44, wherein the patient is also administered one or more immunosuppressing agents.

47. The method of claim 44, wherein the cells have been engineered to reduce or prevent expression of one or more cell surface proteins.


SEQUENCE LISTING (2 sequences)

SEQ ID NO: 1 - DNA, 20 bp, Artificial Sequence
  Description of Artificial Sequence  Synthetic Primer
atggccctgtggatgcgcct

SEQ ID NO: 2 - DNA, 21 bp, Artificial Sequence
  Description of Artificial Sequence  Synthetic Primer
tagttgcagtagttctccagc